SEQUENCE LISTING (2 sequences)

SEQ ID NO: 1 - protein, 193 aa, Homo sapiens [sequence omitted]

SEQ ID NO: 2 - protein, 353 aa, Homo sapiens
MELTELLLVVMLLLTARLTLSSPAPPACDLRVLSKLLRDSHVLHSRLSQCPEVHPLPTPVLLPAVDFSLGEWKTQMEETKAQDILGAVTLLLEGVMAARGQLGPTCLSSLLGQLSGQVRLLLGALQSLLGTQLPPQGRTTAHKDPNAIFLSFQHLLRGKVRFLMLVGGSTLCVRRAPPTTAVPSRTSLVLTLNELPNRTSGLLETNFTASARTTGSGLLKWQQGFRAKIPGLLNQTSRSLDQIPGYLNRIHELLNGTRGLFPGPSRRTLGAPDISSGTSDTGSLPPNLQPGYSPSPTHPPTGQYTLFPLPPTLPTPVVQLHPLLPDPSAPTPTPTSPLLNTSYTHSQNLSQEG

(19) United States
(12) Patent Application Publication
(10) Pub. No.: US 2010/0120663 A1
(43) Pub. Date: May 13, 2010

(54) METHOD FOR INDUCING THROMBOPOIESIS

(76) Inventors: Abina Amine, La Garenne Colombes (FR); William Vainchenker, Paris (FR); Hana Raslova, Le Coudray Montceaux (FR); Machadiya Muslmani, Bretigny S/Orge (FR)

Correspondence Address: BUCHANAN, INGERSOLL & ROONEY PC, POST OFFICE BOX 1404, ALEXANDRIA, VA, 22313-1404, US

(21) Appl. No.: 12/449,161

(22) PCT Filed: Jan. 25, 2008
(86) PCT No.: PCT/EP2008/050902

§ 371 Date: Dec. 8, 2009

Related U.S. Application Data
(60) Provisional application No. 60/897,232, filed on Jan. 25, 2007.

(51) Int. Cl. A61K 38/16; C12N 5/02

(52) U.S. Cl. 514/8; 435/375

(57) ABSTRACT

A regime or regimen for inducing the differentiation of multilineage myeloid progenitor cells (CFU-GEMM) into megakaryocyte progenitor cells and, optionally, into megakaryocytes, that are able to produce platelets both in vivo and in vitro, includes a step a) of administering a thus effective amount of erythropoietin, derivative or agonist thereof to multilinear myeloid progenitor cells.

This application claims the priority of the provisional application U.S. 60/897,232 filed on Jan. 25, 2007, which is incorporated herein by reference.

### FIELD OF THE INVENTION

The present invention relates to the field of biology, more precisely to a method for inducing the differentiation of multilineage myeloid progenitor cells (CFU-GEMM) into megakaryocyte progenitor cells and, optionally, into megakaryocytes, that are able to produce platelets both in vivo and in vitro. The present invention also relates to the applications of such a method in therapy and in cellular biology.

### BACKGROUND OF THE INVENTION

Studies on the regulation of megakaryocytopoiesis have been largely dominated by the concept of a humoral regulation by a humoral factor called thrombopoietin (TPO) which would be, for the megakaryocyte (MK) lineage, the equivalent of erythropoietin (Epo) for the erythroid lineage. The breakthrough in isolating TPO and cloning of its cDNA stems is in the isolation of the murine myeloproliferative leukemia virus (v-mpl). This virus contains a truncated form (v-mpl oncogene) of an orphan cytokine receptor, (c-mpl). It was then shown that MPL was essentially expressed in the MK/platelet lineage and that its in vitro knock-down led to impaired megakaryocyte colony formation in the presence of plasma enriched in megakaryocyte colony stimulating factors. The MPL-ligand was subsequently identified and the protein had all the characteristics of a physiological humoral regulator of platelet production and thus was called thrombopoietin (TPO). In vitro, TPO is a potent stimulator for MK progenitors proliferation, maturation and platelet production. However its effect can be replaced by a combination of cytokines. In vivo studies have shown that daily administration of TPO or overexpression of TPO by gene transfer induces a marked thrombocytosis up to 10-fold the normal number, a result which has never been obtained with other cytokines. However, TPO^{-/-} and c-mpl^{-/-} mice exhibit a marked but non-lethal thrombocytopenia with about ⅕ to 1/10 the normal platelet count. This residual platelet level in the absence of TPO indicates that TPO is not the sole regulator to be involved in platelet production. This demonstrates that there exits other physiological regulators of megakaryopoiesis than TPO. In this respect, the common opinion in the field was that a platelet differentiation residual factor exists and plays a key role when TPO is not active. However, despite their large efforts, the scientists have never been able to undeniably identify this putative residual factor.

“Erythropoietin” (EPO) is a glycoprotein hormone which in humans has a molecular weight of about 30-34 kDa. The mature protein has about 166 amino acids, and the oligosaccharide residues comprise about 40% of the weight of the molecule.

For many years, the only clear physiological role of erythropoietin had been its control of the production of red blood cells (erythropoiesis). Recently, several lines of evidence suggest that erythropoietin, as a member of the cytokine superfamily, performs other important physiologic functions which are mediated through interaction with the erythropoietin receptor (erythropoietin-R). These actions include mitogenesis, modulation of calcium influx into smooth muscle cells and neural cells, production of erythrocytes, hyperactivation of platelets, production of thrombocytes, and effects on intermediary metabolism. It is believed that erythropoietin provides compensatory responses that serve to improve hypoxic cellular microenvironments as well as modulate programmed cell death caused by metabolic stress.

Some authors have suggested that EPO may also be involved in megakaryopoiesis. However, the role of EPO in megakaryocyte differentiation has always been a subject of controversy. Initially it has been shown that EPO both in the human and in mice could give rise to MK colonies. However this result was not confirmed by other teams. Subsequent studies have suggested that EPO in vitro could facilitate MK maturation and proplatelet formation. Nevertheless, these results were still controversial with divergent result and in some reports EPO was even shown to inhibit megakaryocyte maturation (MC DONALD et al., Large, Chronic Doses of Erythropoietin cause Thrombocytopenia in mouse, BLOOD, Vol 80, no 2 (July 15), 1992: pp 352-358; HOMONCIK, Erythropoietin treatment is associated with more severe thrombocytopenia in patients with chronic hepatitis C undergoing antiviral therapy, Am J Gastroenterol. 2006 October; 101(10):2275-82). In vivo EPO has been largely used in the treatment of anemia: Overall it appears that moderate EPO stimulation induces a transient increase in platelet production. In contrast a high stimulation induces a thrombocytopenia by inhibiting platelet production. It has been suggested that this was due to an effect on hematopoietic stem cells by favoring erythroid specification at the expanse of the megakaryocytic lineage.

Since EPO−/− models are not viable, no one could establish that EPO is indeed involved in megakaryopoiesis through its receptor. For the first time, the Inventors succeeded in inducing a functional inactivation of the endogenous EPO in TPO−/− models enabling them to demonstrate the role of EPO in megakaryopoiesis.

Finally, the fact (i) that EPO is the regulatory factor involved in platelet differentiation, alternatively or complementarily to TPO, and (ii) that this EPO function is mediated by interaction with the erythropoietin receptor, is shown here for the first time by the Inventors.

### SUMMARY OF THE INVENTION

The present invention relates to a method for inducing thrombopoiesis comprising the step of administering an effective amount of erythropoietin, derivative or agonist thereof, to a cell type chosen in the group consisting of: multilineage myeloid progenitor cells, megakaryocyte progenitors and megakaryocytes.

More particularly, the present invention relates to a method for inducing the differentiation of multilineage myeloid progenitor cells (CFU-GEMM) into megakaryocyte progenitor cells comprising the step of administering an effective amount of erythropoietin or derivatives to multilineage myeloid progenitor cells.

Advantageously, the method of the present invention enables one to further induce the differentiation of megakaryocyte progenitor cells into megakaryocytes and activate them through phosphorylation of STAT5 to produce platelet both in vitro and in vivo.

Thus, the method of the present invention also enables one to further produce platelets from megakaryocytes.

In a preferred embodiment, the method of the invention further comprises the step of administering to said cell type chosen in the group consisting of: multilineage myeloid progenitor cells, to megakaryocyte progenitors and to megakaryocytes, an effective amount of thrombopoietin or derivatives.

In a particularly preferred embodiment, the method of the invention further comprises the step of administering to said multilineage myeloid progenitor cells an effective amount of thrombopoietin or derivatives.

In another preferred embodiment, the method of the invention is an in vitro method.

In still another preferred embodiment, the method of the invention is an in vivo method for treating and/or preventing thrombocytopenia in a subject.

### BRIEF DESCRIPTION OF THE DRAWINGS

FIG. 1 shows the immunogenicity profile of Rat EPO precursor.

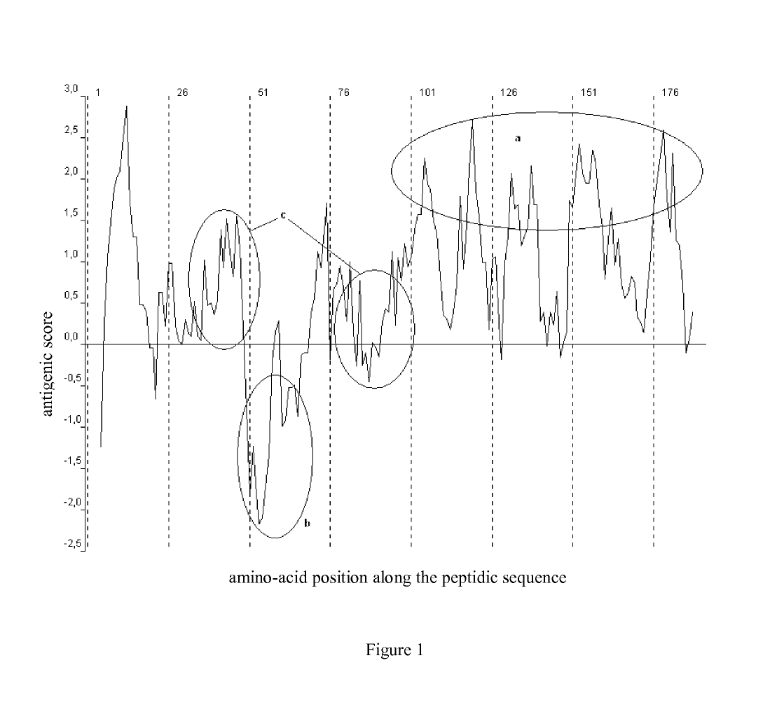

FIG. 2 shows the number of colonies of granulocyte macrophage (CFU-GM), of erythroid progenitor cells (CFU-E) and of megakaryocytes progenitors (Progs-MK) obtained in control and Ad-EPOMut rats.

FIG. 3 shows the western-blot obtained with antibodies directed against STATS (STAT5 total) and phosphorylated STAT5 (P-STAT5) on megakaryocyte derived from human CD34⁺ cells. These cells were cultured for 10 days in presence of thrombopoietin (TPO). They were overnight deprived of cytokines and after they remained unstimulated (control) or stimulated for 10 minutes to 1 hour by 10 UI/ml erythropoietin (EPO) or 100 ng/ml thrombopoietin (TPO).

FIG. 4 shows the percentage of proplatelet forming megakaryocytes with different cytokines. Megakaryocytes were derived from CD34⁺ cells and cultured in the presence of TPO for 10 days. Megakaryocytes were sorted on the coexpression of the CD41 and CD42 antigens and cultured for 48 h either in the absence of cytokine (rien) or in the presence of TPO alone, (TPO), TPO plus EPO, EPO alone (1 U/ml), EPO alone (10 U/ml), SCF alone and IL3 alone

### DETAILED DESCRIPTION

The Inventors have now discovered that the functional inactivation of endogenous erythropoietin induced a dramatic decrease in megakaryocyte progenitors and that erythropoietin plays a critical role in the differentiation of multilineage myeloid progenitor cells (CFU-GEMM) into megakaryocyte progenitor cells, and optionally in turn into megakaryocytes, that are able to produce platelets both in vivo and in vitro. The Inventors have further discovered that this EPO action was mediated by the erythropoietin receptor (EPO-R) activation. STAT5 phosphorylation after EPO supplementation, in absence of TPO, is the signature of this mechanism of action. In vitro and in vivo platelet production further demonstrated that this activation is responsible for the platelet production in wild type animals and recovery in TPO−/− mice.

Consequently, the present invention relates to a method for inducing thrombopoiesis comprising the step a) of administering an effective amount of erythropoietin, derivative or agonist thereof, to a cell type chosen in the group consisting of: multilineage myeloid progenitor cells, megakaryocyte progenitors and megakaryocytes.

Particularly, the present invention relates to a method for inducing the differentiation of multilineage myeloid progenitor cells (CFU-GEMM) into megakaryocyte progenitor cells comprising the step a) of administering an effective amount of erythropoietin, derivative or agonist thereof to multilineage myeloid progenitor cells.

As used herein, “thrombopoiesis” refers to the metabolic pathway of differentiation. from CFU-GEMM to platelets. Thus, thrombopoiesis as used herein includes all the steps or at least one step of said metabolic pathway, involving the differentiation of CFU-GEMM in megakaryocyte progenitor cells, which in turn differentiate in megakaryocytes, which in turn produce platelets.

As used herein “multilineage myeloid progenitor cells (CFU-GEMM)” refers to colony forming units granulocyte/erythroid/macrophage/megakaryocyte, which are well known from one of skill in the art. As an example such cells can be obtained by the culture of CD34+ cells from cord blood or bone marrow under culture conditions well known from the skilled person, such as the conditions described in the examples or in TIANG et al. (Stem Cells, vol. 16, p: 193-9, 1998).

As used herein “megakaryocyte progenitor cells” refers to Colony forming unit-megakaryocytes (CFU-MK), which are well known from one of skill in the art. Such megakaryocyte progenitor cells (CFU-MK) are obtained by differentiation of multilineage myeloid progenitor cells (CFU-GEMM) and give rise to megakaryocytes (MK) after differentiation.

Advantageously, the method of the present invention enables one to further induce the differentiation of megakaryocyte progenitor cells into megakaryocytes.

Advantageously, the method of the present invention enables one to further induce the production of platelets from megakaryocyte cells.

Erythropoietin is well known from one of skill in the art. As an example, the sequence of human erythropoietin has the sequence SEQ ID NO:1.

As used herein, “erythropoietin derivative” refers to a polypeptide having a percentage of identity of at least 60% with erythropoietin or fragment thereof, preferably of at least 75%, as an example of at least 85%, and more preferably of at least 95%.

Said erythropoietin derivative can occupy at least one cellular receptor of erythropoietin, and has the same effect than erythropoietin on differentiation of multilineage myeloid progenitor cells (CFU-GEMM) into megakaryocyte progenitor cells, and optionally in turn into megakaryocytes, that are able to produce platelets both in vitro and in vivo.

As used herein “fragments” refers to polypeptides having a length of at least 25 amino acids, preferably at least 50 amino acids and more preferably of at least 100 amino acids.

As used herein, “percentage of identity” between two amino acids sequences, means the percentage of identical amino-acids, between the two sequences to be compared, obtained with the best alignment of said sequences, this percentage being purely statistical and the differences between these two sequences being randomly spread over the amino acids sequences. As used herein, “best alignment” or “optimal alignment”, means the alignment for which the determined percentage of identity (see below) is the highest. Sequences comparison between two amino acids sequences are usually realized by comparing these sequences that have been previously align according to the best alignment; this comparison is realized on segments of comparison in order to identify and compared the local regions of similarity. The best sequences alignment to perform comparison can be realized, beside by a manual way, by using the global homology algorithm developed by SMITH and WATERMAN (Ad. App. Math., vol. 2, p: 482, 1981), by using the local homology algorithm developed by NEDDLEMAN and WUNSCH (J. Mol. Biol., vol. 48, p: 443, 1970), by using the method of similarities developed by PEARSON and LIPMAN (Proc. Natl. Acd. Sci. USA, vol. 85, p: 2444, 1988), by using computer softwares using such algorithms (GAP, BESTFIT, BLAST P, BLAST N, FASTA, TFASTA in the Wisconsin Genetics software Package, Genetics Computer Group, 575 Science Dr., Madison, Wis. USA), by using the MUSCLE multiple alignment algorithms (Edgar, Robert C., Nucleic Acids Research, vol. 32, p: 1792, 2004). To get the best local alignment, one can preferably used BLAST software, with the BLOSUM 62 matrix, or the PAM 30 matrix. The identity percentage between two sequences of amino acids is determined by comparing these two sequences optimally aligned, the amino acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

As used herein amino acids sequences having a percentage of identity of at least 60%, preferably at least 75%, as an example at least 85%, and most preferably at least 95% after optimal alignment, means amino acids sequences having with regard to the reference sequence, modifications such as substitutions as described in patent applications EP 1737888 and EP 1736481, deletions as the fragments described in patent application US2006/270831, conjugates as described in patent applications US2006/287224, US2006/276634 and EP 106495.

As used herein, the term “EPO agonist” refers to a compound, which. can occupy at least one cellular receptor of erythropoietin, and having the same effect than erythropoietin on differentiation of multilineage myeloid progenitor cells (CFU-GEMM) into megakaryocyte progenitor cells, and optionally in turn into megakaryocytes, which that are able to produce platelets both in vivo and in vitro. Moreover, the term “EPO agonist” is to be understood to also encompass “EPO mimetics” or modifications of full-length rhEpo that prolong plasma survival as described in BLOOD (FRANKLIN BUNN, New agents that stimulate erythropoeisis, BLOOD, 2007; 1009:868-873).

As an example of such potential agonists, one can cite the agonists described in U.S. Pat. No. 5,767,078 and in U.S. Pat. No. 5,835,382.

It is essential to understand that the present invention provides a method for inducing platelet differentiation by stimulating the native erythropoietin receptor EPO-R. Indeed, it is clearly shown here for the first time that this receptor is involved in platelet differentiation. As a consequence, in the method of the invention, induction of target cell differentiation can be performed by using either native EPO, or, and more importantly, any EPO derivative, mimetic or agonist that is able to bind and stimulate the EPO-R.

The EPO derivative or agonist according to the invention is able to occupy at least one receptor of erythropoietin and has the same effect than erythropoietin on thrombopoeisis, in particular on differentiation of multilineage myeloid progenitor cells (CFU-GEMM) into megakaryocyte progenitor cells, optionally in turn into megakaryocytes, that are able to produce platelets both in vivo and in vitro. In other words, the function of said EPO derivative or agonist is mediated by interaction with the EPO receptor.

As an illustration, an EPO derivative that can be used for the purpose of the present invention is described in BLOOD (FRANKLIN BUNN, New agents that stimulate erythropoeisis, BLOOD, 2007; 1009:868-873). This EPO derivative, named CERA (Continuous Erythropoeitin Receptor Activator) is a 60 kDa molecule, which can bind to the EPO receptor more slowly than EPO and has a faster dissociation rate. Thus, CERA can trigger the EPO transduction cascade without being internalized and has a more sustained biologic activity. CERA is thus a good candidate for the method according to the present invention.

EPO-R agonists can be selected among epoetin alpha, kappa, beta, delta or omega;.

Other Epo-R agonists that prolong plasma survival can also be used, as Epo with additional N-linked glycosylation sites into the Epo polypeptide, as an example one can cite the darbepoetin alpha, other modifications can include, but not limited to, synthetic erythropoiesis protein (SEP) with a negatively charged noncarbohydrate precision-length branched polymers attached to this protein at 2 sites; recombinant dimeric Epo linked via a flexible peptide bridge; rhEpo chemically crosslinked via free slfhydryl groups to generate dimers and trimers; modified rhEpo to allow efficient oral administration; rhEpo with a sustained-release formulation; rhEpo modified by site-specific pegylation or immunofusion proteins; erythropoietin-hybrid Fc fusion protein or other engineered ligands that bind Fc receptors; carboxyl terminal peptide (CTP) amino acid sequence fusion to rhEpo; rhEpo or Epo-mimetics coupled to a macromolecular carrier or a biocarrier; for example the albumin protein; fusion of the rhEpo to a biocarrier uses a non-peptidyl polymeric link to form a site-specific conjugate with active peptide/protein drug; hyperglycosylated analogue of darbepoetin-a; antibody or antibody-fusion protein, acting as an erythropoietin receptor agonist; rhEpo modified by addition of polyethylene glycol (PEG) to Epo sugar chains; rhEpo modified by addition of sialic acid-containing carbohydrate chains.

Epo-R activation can also be performed by gene therapy using vectors encoding an Epo-R agonist or gene-activated erythropoietin capable of Epo-R stimulation.

Other examples of derivatives and agonists which can be used for the purpose of the present invention can be found in BLOOD (FRANKLIN BUNN, New agents that stimulate erythropoeisis, BLOOD, 2007; 1009:868-873).

In a preferred embodiment, the method of the present invention further comprises the step b) of administering to said cell type chosen in the group consisting of: multilineage myeloid progenitor cells, megakaryocyte progenitors and megakaryocytes, an effective amount of trombopoietin, derivative or agonist thereof.

Particularly, the method of the invention further comprises the step b) of administering to said multilineage myeloid progenitor cells an effective amount of thrombopoietin, derivative or agonist thereof.

Said administering step b) can operate before, simultaneously or after the administering step a).

Thrombopoietin is also well known from one of skill in the art and, as an example, the sequence of human thrombopoietin has the sequence SEQ ID NO:2.

As used herein, “thrombopoietin derivative” refers to a polypeptide having a percentage of identity of at least 60% with thrombopoietin or fragment thereof, preferably of at least 75%, as an example of at least 85%, and more preferably of at least 95%.

Said thrombopoietin derivative can occupy at least one cellular receptor of thrombopoietin, and having the same effect than thrombopoietin on differentiation of multilineage myeloid progenitor cells (CFU-GEMM) into megakaryocyte progenitor cells, and optionally in turn into megakaryocytes, that are able to produce platelets both in. vivo and in vitro.

As used herein “fragments” refers to polypeptides having a length of at least 25 amino acids, preferably at least 50 amino acids and more preferably of at least 100 amino acids.

As used herein amino acids sequences having a percentage of identity of at least 60%, preferably at least 75%, as an example at least 85%, and most preferably at least 95% after optimal alignment, means amino acids sequences having with regard to the reference sequence, modifications such as substitutions as described in patent application US2006/160995, deletions as the fragments described in patent application JP2006232697, conjugates as described in international patent application PCT WO 00/00612.

As used herein, the term “TPO agonist” refers to a compound, which can occupy at least one cellular receptor of thrombopoietin, and having the same effect than thrombopoietin on differentiation of multilineage myeloid progenitor cells (CFU-GEMM) into megakaryocyte progenitor cells, and optionally in turn into megakaryocytes, that are able to produce platelets both in vivo and in vitro.

As an example of such potential agonist, one can cites the agonists described in International patent application PCT WO 01/07423 and in patent U.S. Pat. No. 6,887,890.

According to another preferred embodiment, the method of the invention is an in vitro method.

As such the method of the invention can be realized on multilineage myeloid progenitor cells obtained by CD34+ or CD34− (immature progenitor cells) cells separation on cord. blood, bone marrow and cytapheresis, or by culture and differentiation of specific cell lines such as embryonic cells (ES cells), amniotic cells or dedifferentiated fibroblast cells.

One of skill in the art can simply determined the effective amount of EPO, derivative or agonist thereof eventually in combination with TPO, derivative or agonist thereof in order to obtain the differentiation of multilineage myeloid progenitor cells into megakaryocyte progenitor cells, and optionally in turn into megakaryocytes, that are able to produce platelets both in vivo and in vitro.

As an example, an effective amount of erythropoietin for inducing the differentiation of multilineage myeloid progenitor cells into megakaryocyte progenitor cells and optionally in turn into megakaryocytes, that are able to produce platelets both in vivo and in vitro, is comprised between 0.1 and 100 U/ml, preferably between 0.5 and 50 U/ml, and more preferably between 1 and 10 U/ml.

As another example, an effective amount of thrombopoietin for inducing the differentiation of multilineage myeloid progenitor cells into megakaryocyte progenitor, and optionally in turn into megakaryocytes, that are able to produce platelets both in vivo and in vitro, is comprised between 10 and 10,000 U/ml, preferably between 15 and 5,000 U/ml, and more preferably between 5 and 2,000 U/ml.

According to still another preferred embodiment, the method of the invention is a method for treating and/or preventing thrombocytopenia in a subject.

As used herein, the term “subject” refers to a vertebrate, preferably a mammal, and most preferably a human.

As used herein, “thrombocytopenia”, also called “thrombopenia” refers to a disease associated with a decrease in platelet count. Thrombocytopenia can results from vitamin B12 or folic acid deficiency, leukaemia or myelodysplastic syndrome, idiopathic thrombocytopenic purpura (ITP), thrombotic thrombocytopenic purpura (TTP), haemolytic-uremic syndrome (HUS), systemis lupus erythematosus (SLE), cirrhosis, different cancers, hepatic failure, viral infection such as HIV or can be medication-induced such with anticancer drugs, quinine or abciximab.

Preferably, said thrombocytopenia is chosen among a single lineage cytopenia and bicytopenia.

As used herein, the “single lineage cytopenia” refers to an. isolated cytopenia.

As used herein, “bicytopenia” refers to a decrease in two or three cell lineage selected among white blood cells, platelet and red blood cells.

Preferably, bicytopenia refers to a decrease in platelet and red blood cells.

The “effective amount of erythropoietin, derivative or agonist thereof” required for inducing the differentiation of multilineage myeloid progenitor cells (CFU-GEMM) into megakaryocyte progenitor cells and optionally in turn into megakaryocytes, that are able to produce platelets both in vivo and in vitro, can be determined by routine experiments by one of skill in the art.

The inventors have surprisingly established that said effective amount is far less than the effective amount of erythropoietin, derivative or agonist thereof required for inducing erythroid differentiation.

As an example, such effective amount of EPO, derivative or agonist thereof is comprised between 2,000 and 20,000 units per month, preferably between 4,000 and 10,000 units per month.

The administration of erythropoietin, derivative, or agonist thereof and eventually of thrombopoietin, derivative, or agonist thereof is performed via a technique selected in the group consisting in intravenous injection, intravaginal injection, intrarectal injection, intramuscular injection, subcutaneous, intradermic injection, orale or nasal delivery. Preferably, the administration is performed via subcutaneous, intradermic, or intramuscular injection. Single injection or multiple injections at the same or at different loci can be performed.

Erythropoietin, derivative, or agonist thereof and eventually thrombopoietin, derivative, or agonist thereof is formulated for short or long term delivery. For example, the formulation allows a sustained or immediate release of the active principle.

Another aspect of the invention concerns the use of erythropoietin, derivative or agonist thereof, optionally in combination of thrombopoietin, derivative or agonist thereof, for the preparation of a medicament for treating or preventing thrombocytopenia in a subject.

In still another aspect, the invention concerns the use of erythropoietin, derivative or agonist thereof as an in vitro agent for inducing the differentiation of multilineage myeloid progenitor cells (CFU-GEMM) into megakaryocyte progenitor cells, and optionally in turn into megakaryocytes, that are able to produce platelets.

Advantageously, said in vitro agent for inducing the differentiation of multilineage myeloid progenitor cells (CFU-GEMM) into megakaryocyte progenitor cells, and optionally in turn into megakaryocytes, that are able to produce platelets, further comprises thrombopoietin, derivative or agonist thereof.

The practice of the invention employs, unless other otherwise indicated, conventional techniques or protein chemistry, molecular virology, microbiology, recombinant DNA technology, and pharmacology, which are within the skill of the art. Such techniques are explained fully in the literature. (See AUSUBEL et al., Current Protocols in Molecular Biology, Eds., John Wiley & Sons, Inc. New York, 1995; Remington's Pharmaceutical Sciences, 17^{th} ed., Mack Publishing Co., Easton, Pa., 1985; and SAMBROOK et al., Molecular cloning: A laboratory manual 2^{nd} edition_ Cold Spring Harbor Laboratory Press-Cold Spring Harbor, N.Y., USA, 1989).

Unless defined otherwise, all technical and scientific terms used herein have the same meaning as is commonly understood by one of the skill in the art to which this invention belongs.

The present invention may be better understood by reference to the following non-limiting Examples, which are provided as exemplary of the invention. The following examples are presented in order to more fully illustrate the preferred embodiments of the invention. They should. in no way be construed, however, as limiting the broad scope of the invention.

### Examples

1) Design of an EPO Mutant:

The mature erythropoietin consists of 166 amino acid residues, along with 3 oligosaccharide chains. The protein itself has a mass of 18 KDa while the human glycosylated protein has a mass of 30 KDa. This mature erythropoietin is obtained from an EPO precursor being composed of one main domain EPO (26-192) which is a four helix cytokine domain that is involved in the erythrocyte differentiation and the maintenance of a physiological level of circulating erythrocyte mass, and the peptide-signal domain (1-26).

We performed an analysis of immunogenicity of Rat EPO precursor by using antigenic prediction described by WELLING et al. (FEBS, vol. 188 (2), page 215-218, 1985). Said immunogenicity analysis has revealed an average inmiunogenic protein with several picks of immunogenicity, with a large area between 110 and 185 being highly antigenic, a region between 45 and 65 showing very low antigenic values and two regions which are in between these values.

An erythropoietin derivative has been generated by introducing modifications in order to create new antigenic regions able to generate a cross reactive antibody response targeting the endogenous EPO. The comparison of the immunogenicity profiles of said derivative with rat EPO revealed a strong immunogenicity increase in few regions.

Then, we have constructed an Adenovirus coding for the designed erythropoietin derivative (Ad-EPOMut).

2) Construction of Recombinant E1-E3 Deleted Adenovirus Vectors:

Fragments of nucleotides coding for the EPOmut, EPOmut1 were cloned in an adenoviral shuttle vector CAG pShuttle (ADDGENE) in which the homologous EPO sequence is under the control of the CAG promoter (i.e., a combination of chicken beta-actin promoter and cytomegalovirus immediate-early enhancer) and the SV40 polyadenylation signal for obtaining the CAG pShuttle.EPOhom, or CAG pShuttle.EPOhom1 plasmids.

The CAG pShuttle.EPOhom or CAG pShuttle.EPOhom1 plasmids were then digested by Pme I (BIOLABS) and a homologous recombination with the pAdEasy-1 adenoviral backbone plasmid (STRATAGENE) was carried out in Escherichia Coli for the generation of the recombinant adenovirus Ad-EPOMut, Ad-EPOMut1. An Ad.RSV.nul1 or Ad.CAGnul1 generated by recombination between pAdEasy-1 and the pShuttle.RSV or pShuttle.CAG, was used as a control.

3) Induction of a Functional Inactivation of the Endogenous EPO Protein in Rat After Vaccination with the Mutant EPO

20 females OFA rats (Sprague Dawley), 8 weeks old were obtained from Charles River, France, and housed at the CERFE animal facility (Genopole, Evry, France). All experiments were conducted in concordance with the local ethical and scientific policies.

All animals were bred in negative pressure isolators, placed in confined room until downgrading was validated (absence of infectious particles in animal blood), and then were acclimatized for 10 days before the beginning of procedures.

Rats were individually labelled for this study, and all manipulations in presence of adenovirus particles were made in a confined room in compliance with the French GMO regulations.

The 10 weeks animals were subdivided in 3 groups: PBS, AdNull (control recombinant adenovirus without transgene-encoding sequence) and Ad-EPOMut treated group (recombinant adenovirus coding for the EPO derivative).

Isoflurane-anesthezied animals were subcutaneously injected with 1.10⁹ infectious particles of Ad-EPOMut, AdNull in 100 μl of PBS (Gibco, Invitrogen) or PBS alone using a 18 G needle gauge.

Animals have been observed every week, and monthly followed by a blood cells numeration. The blood samples were obtained by puncture of the retro-orbital sinus using a Pasteur pipette. 1 ml of blood was collected per rat once a month.

For the peripheral blood hematologic measurements, 300 μl of the 1 ml collected blood was dropped immediately in a 1.5 ml polypropylene tube containing 3 μl of EDTA 0.5M (Gibco) for haematological measurement. The measurement was made between 30 minutes and 4 hours after the blood puncture using the MS9 animal blood counter (MELET SCHLOESING).

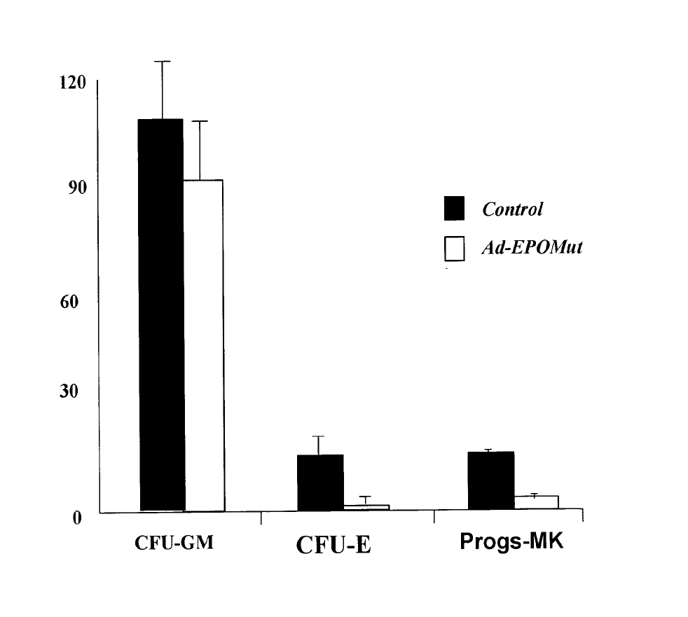

The rest of blood was centrifuged in a 1,5 ml polypropylene tube at 21° C. between 30 min and 1 h after the puncture, 12 minutes at 2000 rpm. Sera were collected carefully with a p200 tip, stocked in a 1.5 ml propylene tube and immediately stored at −20° C. The number of White Blood Cell (WBC), and Red Blood Cell (RBC), the value of hematocrit (HCT) and the number of Platelet (PLT) have been determined for each blood sample. The results shown in table 1 demonstrate the full EPO protein neutralization in rats with a fast reduction in red blood cells in responding rats. Animals injected with PBS or AdNull had normal hematologic counts. Two of the three EPO-KO rats had also a decrease in platelet counts. These results are correlated with a massive megakaryocyte progenitors reduction as shown in FIG. 2.

TABLE 1
blood cell numeration of rat treated with Ad-EPOMut, AdNull or PBS during 3 months.

| | | 1 MONTH | | | | 2 MONTHS | | | | 3 MONTHS | | | |
|---|---|---|---|---|---|---|---|---|---|---|---|---|---|
| | | WBC | RBC | HCT | PLT | WBC | RBC | HCT | PLT | WBC | RBC | HCT | PLT |
| | N° | (10³/μl) | (10⁶/μl) | (%) | (10³/μl) | (10³/μl) | (10⁶/μl) | (%) | (10³/μl) | (10³/μl) | (10⁶/μl) | (%) | (10³/μl) |
| PBS | 1 | 7.1 | 10.75 | 59.6 | 274 | 7 | 11.04 | 61.6 | 361 | 8.83 | 10.84 | 60.2 | 321 |
| | 2 | 6.29 | 6.87 | 38.7 | 557 | 5.39 | 10.94 | 61.4 | 315 | 6.13 | 7.95 | 43.9 | 296 |
| | 3 | COAG | | | | 9.16 | 10.27 | 57.4 | 526 | 7.01 | 7.48 | 42.6 | 556 |
| | 4 | 3.24 | 8.91 | 50.6 | 448 | 5.79 | 11.28 | 62.8 | 298 | 6.5 | 7.8 | 42.7 | 314 |
| | 5 | 3.59 | 8.4 | 47.7 | 167 | COAG | | | | COAG | | | |
| | mean | | | | 361.5 | | | | 375 | | | | 371.75 |
| AdNull | 6 | 6.68 | 11.67 | 64.5 | 418 | 9 | 10.2 | 55.5 | 624 | 6.32 | 8.1 | 44.5 | 468 |
| | 7 | 7.43 | 9.86 | 55.9 | 538 | 4.54 | 8.63 | 48.3 | 237 | 8.88 | 10.44 | 57.6 | 456 |
| | 8 | 8.4 | 9.07 | 51.8 | 482 | | | | | SACRIF | | | |
| | 9 | 7.8 | 10.81 | 59.3 | 762 | 5.51 | 9.96 | 55.1 | 494 | 7.46 | 8.11 | 45.8 | 689 |
| | 10 | 5.6 | 10.73 | 60.4 | 451 | 7.43 | 9.89 | 56.2 | | SACRIF | | | |
| | 11 | COAG | | | | 6.63 | 9.25 | 50.8 | 602 | 5.54 | 7.59 | 41.3 | 716 |
| | 12 | 7.46 | 8.79 | 49.8 | 1042 | 8.37 | 7.98 | 44.7 | 748 | 7.92 | 9.27 | 51.6 | 753 |
| | mean | | | | 615.5 | | | | 541 | | | | 616.4 |
| Ad- | 13 | 2.43 | 3.71 | 25 | 821 | 1.96 | 0.76 | 4.1 | 128 | SACRIF | | | |
| EPOMut | 14 | 7.1 | 5.19 | 31.3 | 523 | 5.03 | 0.93 | 5 | 411 | SACRIF | | | |
| | 15 | 4.5 | 10.02 | 62.6 | 431 | 0.73 | 0.7 | 4.5 | 54 | SACRIF | | | |
| | 16 | 6.96 | 7.39 | 41.8 | 316 | 6.81 | 5.88 | 35.2 | 359 | 8.39 | 8.03 | 43.6 | 652 |
| | 17 | 11.59 | 10.15 | 62.2 | 1116 | 7.91 | 6.28 | 37.7 | 391 | 7.93 | 8.28 | 46.3 | 486 |
| | mean | | | | 425.4 | | | | 208.6 | | | | 569 |

4) Presence of a Humoral Response Against Endogenous EPO

A ninety six well plate (NUNC MAXISORB) was coated with 50 μl of recombinant rat EPO (SIGMA-ALDRICH) at 10 μg/ml diluted in coating buffer (0.1M carbonate/bicarbonate buffer, pH 9.6) overnight at 4° C.

The plate was washed 3 times with 300 μl per well of PBS containing 0,05% Tween® 20 (SIGMA-ALDRICH), and then saturated with PBS containing 3% BSA (SIGMA-ALDRICH) for 90 minutes at room temperature.

The contents of wells were flicked out and, after 3 washes, 100 μl of a serial dilution of serum from KO EPO rat (Ad-EPOMut treated rats) and negative control rats (AdNull treated rats) was added and incubated for 1 hour at room temperature and 1 hour at 37° C. Serum were diluted in PBS/1.5%BSA from 1/50 to 1/800.

The wells were washed 4 times and incubated with 100 μl of horseradish peroxidase-conjugated goat anti-rat IgG and IgM (JACKSON IMMUNORESEARCH LABORATORIES) diluted at 1/10000 in PBS/BSA 1.5% for 1 hour at room temperature. After 5 washes 100 μl of TMB (BD BIOSCIENCE) was added in each well for 20 minutes in the dark and the reaction was stopped using 50 μl of HCl 1M. The absorbance was measured (μQUANT spectrophotometer, BIO-TEK INSTRUMENTS) 30 minutes after stopping the reaction, at 450 nm against a reference blank at 570 nm.

The results assays have shown that the sera from AdNull did not contain antibodies reactive against endogenous rat EPO by contrast with the sera from Ad-EPOMut which contained antibodies reactive against endogenous rat EPO.

Finally, our results obtained by specific ELISA have also shown. that the antibodies reactive against endogenous rat EPO do not cross react with endogenous TPO demonstrating that the observed decrease of progenitor megakaryocyte cells is specifically related to EPO.

5) Dosage Erythropoietin In Rat Serum

EPO (erythropoietin) levels quantification in KO-EPO rat sera was performed by an ELISA using “Quantikine mouse/rat EPO immunoassay” (R&D system, ref. MEP00) according to the manufacturer's instructions. A polystyrene microtiter plate coated with monoclonal antibodies specific for mouse/rat EPO was incubated with standards, positive control, and samples for 2 hours at room temperature on a horizontal orbital microplate shaker set at 200 rpm. After washing 5 times, an HRP-linked monoclonal antibody specific for mouse/rat EPO is added to the wells for 2 hours. Following washes, a substrate solution is added to the wells during 30 minutes and the enzyme reaction was stopped with Stop Solution. Optical density was assessed at 450 nm and to correct wavelength a measure was performed at 570 nm. The serum EPO levels were then determined using a standard curve by comparing their OD values.

The results are shown in table 2.

TABLE 2
EPO cytokine Detection in KO-EPO rat sera.

| | | OD at 450 nm | [EPO] ng/ml |
|---|---|---|---|
| | positive control | 0.244 | 0.177 |
| 1st month | PBS | 0.017 | 0.040 |
| | AdNull | 0.086 | 0.081 |
| | KO1 | 0.021 | 0.042 |
| | KO2 | 0.019 | 0.041 |
| | KO3 | 0.018 | 0.040 |
| 2nd month | PBS | 0.021 | 0.042 |
| | AdNull | 0.016 | 0.039 |
| | KO1 | 0.020 | 0.042 |
| | KO2 | 0.038 | 0.052 |
| | KO3 | 0.137 | 0.112 |

Values obtains from ELISA at 450 nm and determination of the EPO concentration by the standard curve (ng/ml)

Normally, the serum EPO level exponential increase, as the hematocrit decreases and as hypoxia increases, is very important. The results show that no EPO detectable level was observed in PBS, or AdNull rat, but also for KO-EPO rats as shown in table 2 with most of the values under the minimum detection dose of 0.05 ng/ml despite the hypoxia induced by the complete neutralization of the species differentiation and absence of blood cells.

On the contrary, the two other rats in Ad-EPOMut group (n° 16 and 17, table 1), which show transient and mild neutralized phenotype have presented an OD at 450 nm of 0.801 and 1.139 means 0.515 ng/ml and 0.719 ng/ml of EPO concentration, respectively.

Because of the presence of anti-rat EPO antibodies in serum, the relationship between hematocrit, hypoxia and EPO level is likely to be complex.

Finally, the results have established that the anti-rat EPO antibodies in serum were functionally active, leading to the blockade of the endogenous EPO and its functionality. Even if this anaemia induces a production of EPO in KO-EPO rats, the neutralizing antibodies block this EPO immediately before it interacts with EPO receptor. Furthermore, the results have established that EPO is implicated in platelet production like in red blood cell production, and that this implication seems to be independent.

6) Influence of Ad-EPOMut Injection On Megakaryocyte Progenitor Cells

The numbers of CFU-GM (granulocyte/macrophage progenitors), CFU-MK (megakaryocytes progenitors) and CFU-E (erythroid progenitors) derived colonies were analyzed in three anaemic rats (n° 13, 14, 15) and two negative control rats (n° 7 and 11) two months following the injection.

The results show that the three AdCAG-EPOMut-treated rats had a massive significant reduction of erythroid progenitors (p=0.02) compared to control animals (FIG. 2). Surprisingly, the results also show that the three AdCAG-EPOMut-treated rats had a similar significant reduction of megakaryocyte progenitors (p=0.0005) compared to control animals (FIG. 2).

In contrast the number of CFU-GM-derived colonies were comparable in both groups, with a reduced non significant decrease (p=0.3) of CFU-GM (FIG. 2).

Finally, the results confirm the critical role of EPO for the differentiation of multilineage myeloid progenitor cells (CFU-GEMM) in erythroid progenitor cells but also identify for the first time EPO as critical in the independent differentiation of multilineage myeloid progenitor cells (CFU-GEMM) in megakaryocyte progenitor cells (CFU-MK).

7) Implication of Erythropoietin In Megakaryocytes Progenitors Formation

5.10⁵ cells/ml from bone marrow of Ad-Null and Ad-EPOMut injected rats were cultivated in 12 wells plate with 1 ml of IMDM medium (GIBCO, INVITROGEN) per well supplemented with glutamin/penicillin/streptomycin, alpha-thioglycerol (2 mM/ml, 100 U/ml, 100 μg/ml, 76 μM), 1.5% carbonated BSA (20 ml BSA prepared with 500 μl of sodium carbonate), 5% SVF, 10 μl/ml of insulin-transferrin-selenium (INVITROGEN), 20 μl/ml liposomes and 10 ng/ml hTPO (ABCYS). The cells were then incubated for 9 days at 37° C., 5% CO2, 95% humidity.

Then, the cells were observed by microscope to established differentiation status and counted. The number of megakaryocytic cells after nine days of culture for Ad-Null and Ad-EPOMut injected rats are resumed in Table 3.

| Rat | Cells/ml |
|---|---|
| Ad-Null 1 | 66 10⁶ |
| Ad-Null 2 | 67 10⁶ |
| KO1 | 26 10⁶ |
| KO2 | 20 10⁶ |
| KO3 | 26 10⁶ |

The results show that the KO-rat (Ad-EPOMut injected rats) show three times less megakaryocytic cells than Ad-Null treated rat (see. table 3) confirming the implication of EPO in megakaryocytopoiesis.

The microscope analysis of the obtained megakaryocytic cells shows that the megakaryocytic cells from Ad-Null injected rats were differentiated in mature megakaryocytes with some of them which are differentiated in proplatelet-forming megakaryocytes. Surprisingly, and despite of the presence of thrombopoietin in the culture medium, the megakaryocytic cells from Ad-EPOMut injected rats were not differentiated or not completely differentiated with most cells being megakaryocytic progenitor cells and some as immature megakaryocytes.

Finally, these results confirm the previously identified (cf. 4 and 5) critical role of EPO in megakarocytopoiesis in association with TPO, and established that EPO is critical at all the differentiation steps, from CFU-GEMM to CFU-MK and also from CFU-MK to megakaryocyte and finally to platelet production.

8) A Low Dose of Erythropoietin is Sufficient for Megakaryocytes Progenitor Formation

We have established that the expression of the erythropoietin receptor (EPOR) is increased as a function of megakaryocytes ploidy, which corresponds to a megakaryocytopoiesis differentiation marker.

In order to identify the functionality of EPOR in the formation of megakaryocytes, in vitro experiments have been initiated with human bone marrow CD34+ cells obtained from volunteers.

We established the phosphorylation profile of STAT5 which is implicated in EPO and TPO signalling pathways after stimulation of the cells with EPO or TPO (10 min, 30 min, 1 hour and 2 hours after stimulation).

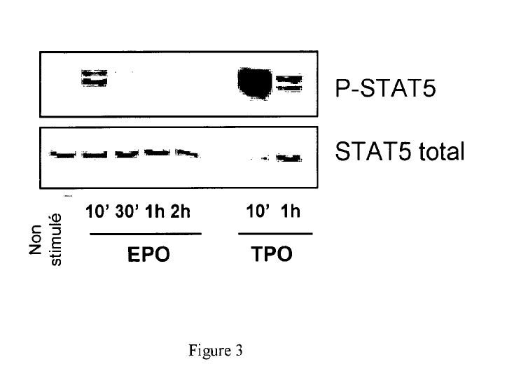

The results have established a clear activation of STAT5 (see western blot of FIG. 3), with the appearance of phosphorylated STAT5, after the stimulation with EPO, even if this stimulation is fewer than those obtained with TPO.

Consequently, these results established the functional expression of EPOR in megakarocitic cells with the activation of STAT5 after EPO stimulation.

In order to better identify the potential role of EPOR in platelet genesis, bone marrow cells have been cultured for 9 days with TPO as described previously, then the liquid medium has been changed to be deprived in TPO and different cytokines conditions have been tested until the day 14 of culture(i.e. EPO (1 U/mL), EPO (10 U/mL), EPO (1 U/mL) and TPO (10 ng/ml), TPO (10 ng/mL), SCF (25 ng/mL), IL-3 (100 U/mL) and control (Mock)). Finally, the percentage of megakaryocytes engaged in thrombopoietic maturation has been evaluated at J14 of culture for the different cytokines conditions.

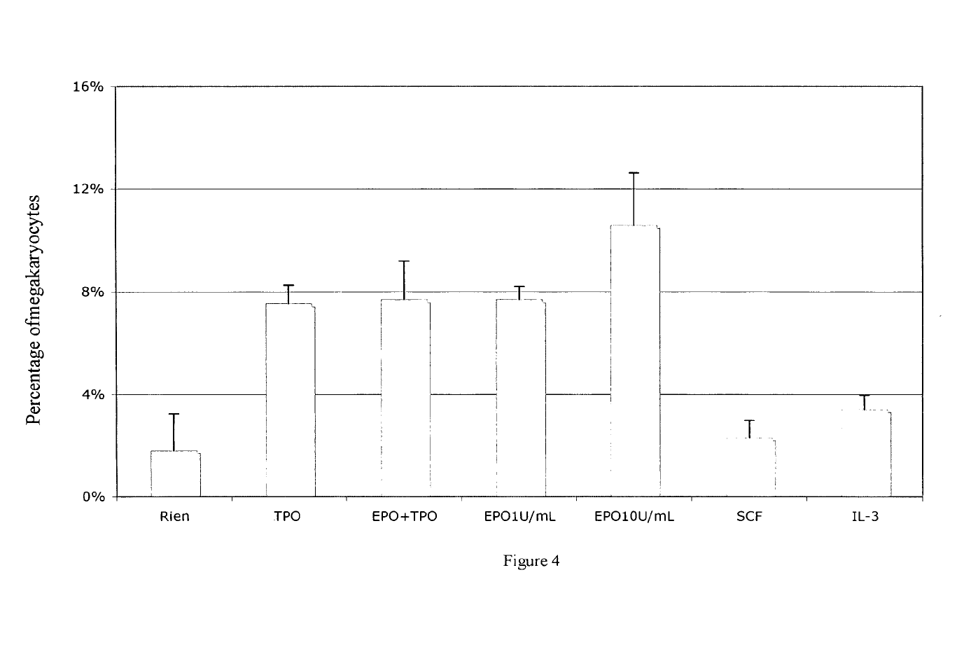

The results shows that EPO alone (EPO 1 U/ml and EPO 10 U/ml) have a similar stimulation role than TPO on the percentage of megakaryocytes in the culture (FIG. 4). Moreover, said EPO concentrations are fewer than those necessary for red blood cell differentiation (between 30 and 50 U/ml). Since, the combination of EPO and TPO has no cumulative effect on said stimulation, it is probable that EPO and TPO are implicated in megakaryocytopoiesis by the same signal pathway.

Finally, these results confirm those obtained with Ad-EPOMut injected rats establishing the implication of EPO in platelet genesis, and more precisely the implication of EPO in megakaryocytopoiesis.

9) Functional Inactivation of the Endogenous EPO Protein in Mice

9.1 Induction of a functional inactivation of the endogenous EPO protein in mice after vaccination with the mutant EPO.

In order to generalize, the induction of functional inactivation of endogenous EPO protein after vaccination with mutants EPO to other species, experiments were conducted on mice strains.

29 C57BL6/J TPO−/− mice, 12-14 weeks old were obtained from Dr. Michèle Souyri (Hospital Paul Brousse—Paris, France). All experiments were conducted in concordance with the local ethical and scientific policies.

All animals were bred in negative pressure isolators, placed in confined room until downgrading was validated (absence of infectious particles in animal blood), and then were acclimatized for 10 days before the beginning of procedures.

For this study mice were individually labelled.

All manipulation in presence of adenovirus particles were made in a confined room in compliance with the French GMO regulations.

Genetically modified and wild type animals were subdivided in 2 groups: Ad-Null (recombinant adenovirus E1- and E3-deleted but free of the transgene sequence) and Ad-EPOmix conditions.

The mix consists of Ad-EPOmut1 and Ad-EPOmut at a ratio of 1:1, and was injected on C57BL6/J mice or C57BL6/J TPO−/−. As a control, mice were injected with the same dose of Ad-Null.

Isoflurane-anesthezied animals were injected with 2.10⁸ or 5.10⁸ infectious particles of Ad-EPOMix or Ad-Null in 100 μl of PBS (Gibco, Invitrogen) using a 25 G needle gauge.

The injections were made at day zero (t=0) for the prime injection by subcutaneous route and at 1.2 month for the boost injection using the same dose by intramuscular route.

Animals were clinically observed every week, and followed by a blood cells numeration once or twice a month. Blood samples were obtained by puncture of the retro-orbital-sinus using a Pasteur pipette.

For the peripheral blood hematologic measurements, 50 μl collected blood was dropped immediately in a 1.5 ml polypropylene tube containing 1 μlof EDTA 0.5M (Gibco) for haematological measurement. The measurement was made between 30 minutes and 4 hours after the puncture using the MS9 animal blood counter (MELET SCHLOESING).

The remaining blood (100 μl) was centrifuged in a 1,5 ml polypropylene tube at 21° C. between 30 min and 1 h after the puncture. Then, blood was centrifuged 12 min at 2000rpm for serum collection, which was recovered carefully with a p200 tip, stocked in a 1.5 ml propylene tube and immediately stored at −20° C.

The number of White Blood Cell (WBC), and Red Blood Cell (RBC), the hematocrit value (HCT) and the number of Platelets (PLA) have been determined for each blood sample. The results are shown in table 4

TABLE 4
blood numeration of TPO−/− c587Bl6/j treated with
Ad-Null or Ad-EPOmix followed during one month.

| | | | 1 month post prime | | |
|---|---|---|---|---|---|
| | dose | | WBC | | platelets |
| Adenovirus | (ip/mouse) | # | 10e+3/mm3 | HCT % | 10e+3/mm3 |
| Ad-EPOmix | 2.10e+8 | 10 | 14.56 | 52.8 | 552.5 |
| Ad-EPOmix | 5.10e+8 | 17 | 8.53 | 74.9 | 511 |
| Ad-EPOmix | 5.10e+8 | 15 | 6.74 | 42.1 | 501 |
| Ad-EPOmix | 2.10e+8 | 12 | 5.98 | 51.7 | 461 |
| Ad-EPOmix | 2.10e+8 | 2 | 5.04 | 43.7 | 454 |
| Ad-EPOmix | 2.10e+8 | 9 | 5.08 | 62.5 | 438 |
| Ad-EPOmix | 2.10e+8 | 6 | 10.64 | 83.8 | 437 |
| Ad-EPOmix | 2.10e+8 | 5 | 8.36 | 45.7 | 409 |
| Ad-EPOmix | 5.10e+8 | 16 | 6.98 | 42.6 | 404 |
| Ad-EPOmix | 2.10e+8 | 8 | 7.98 | 68.9 | 384 |
| Ad-EPOmix | 5.10e+8 | 19 | 7.32 | 42.9 | 376 |
| Ad-EPOmix | 5.10e+8 | 14 | 5.37 | 28.3 | 351 |
| Ad-EPOmix | 2.10e+8 | 7 | 11.06 | 53.7 | 323 |
| Ad-EPOmix | 2.10e+8 | 4 | 6.6 | 59.8 | 317 |
| Ad-EPOmix | 5.10e+8 | 18 | 6.76 | 36.4 | 317 |
| Ad-EPOmix | 5.10e+8 | 20 | 6.8 | 42.3 | 313 |
| Ad-EPOmix | 2.10e+8 | 11 | 7.99 | 78 | 301 |
| Ad-EPOmix | 2.10e+8 | 3 | 11.33 | 51.6 | 263 |
| Ad-EPOmix | 5.10e+8 | 21 | 10.18 | 39.4 | 260 |
| Ad-EPOmix | 2.10e+8 | 13 | 7.64 | 13.4 | 243 |
| Ad-EPOmix | 2.10e+8 | 1 | 9.03 | 43.6 | 225 |
| Ad-Null | 5.10e+8 | 24 | 5.45 | 46.4 | 206 |
| Ad-Null | 5.10e+8 | 23 | 10.5 | 47.2 | 202 |
| Ad-Null | 5.10e+8 | 25 | 8 | 43.1 | 188 |
| Ad-Null | 5.10e+8 | 26 | 6.06 | 46.4 | 171 |

According to results obtained in table 4, platelets normal values for C57BL6/J TPO−/− mice are 191.33 (±10.78).10³ platelets/mm³. We considered values higher than 300.10³ platelets/mm³ as increasing values.

The results obtained during three months with this strain confirm the generation of an EPO-KO phenotype following vaccination with Ad-EPOmix.

C57B16/J TPO−/− mice (TPO−/−) injected with the Ad-EPOMix displayed transient elevations in hematocrit (HCT) during the first month. This high elevation of hematocrit was followed by a profound anaemia the next months. The number of White Blood Cell stays normal in both treated groups.

The first month post prime injection, HCT varies with results showing early decreasing in some mice and increasing or normal HCT values in others (see table 4). 2 mice presented an early anemia, with 13.4% and 28.3% HCT levels.

As seen in Table 4, Ad-Null injection has no influence on platelets variations. In the Ad-EPOmix group, 17 out of 21 (80.95%) showed a platelets level higher than 300.10³ PLA/mm³, 9 out of 21 (42.85%) had more than 400.10³ PLA/mm³ platelets (mean 463.33.10³ PLA/mm³), and 3 mice showed platelet values higher than 500.10³ PLA/mm3. This group had a 2.4 fold increase when compared to the Ad-Null treated group.

Table 5 shows the results obtained from mice having normal hematocrit at 1 month. If we compare Ad-Null and Ad-EPOmix treated group showing an identical HCT we observed that 9 out of 11 (81.8%) C57B16/J TPO−/− mice treated with Ad-EPOmix had more than 300.10⁸/mm3 platelet values.

TABLE 5
Blood numeration in TPO−/− mice treated with Ad-EPOmix
or Ad-Null within the first month post injection.

| | | | 1 month post prime | | |
|---|---|---|---|---|---|
| | dose | | WBC | | platelets |
| Adenovirus | (ip/mouse) | # | 10e+3/mm3 | HCT % | 10e+3/mm3 |
| Ad-Null | 5.10e+8 | 26 | 6.06 | 46.4 | 171 |
| Ad-Null | 5.10e+8 | 25 | 8 | 43.1 | 188 |
| Ad-Null | 5.10e+8 | 23 | 10.5 | 47.2 | 202 |
| Ad-Null | 5.10e+8 | 24 | 5.45 | 46.4 | 206 |
| Ad-EPOmix | 2.10e+8 | 1 | 9.03 | 43.6 | 225 |
| Ad-EPOmix | 2.10e+8 | 3 | 11.33 | 51.6 | 263 |
| Ad-EPOmix | 5.10e+8 | 20 | 6.8 | 42.3 | 313 |
| Ad-EPOmix | 2.10e+8 | 7 | 11.06 | 53.7 | 323 |
| Ad-EPOmix | 5.10e+8 | 19 | 7.32 | 42.9 | 376 |
| Ad-EPOmix | 5.10e+8 | 16 | 6.98 | 42.6 | 404 |
| Ad-EPOmix | 2.10e+8 | 5 | 8.36 | 45.7 | 409 |
| Ad-EPOmix | 2.10e+8 | 2 | 5.04 | 43.7 | 454 |
| Ad-EPOmix | 2.10e+8 | 12 | 5.98 | 51.7 | 461 |
| Ad-EPOmix | 5.10e+8 | 15 | 6.74 | 42.1 | 501 |
| Ad-EPOmix | 2.10e+8 | 10 | 14.56 | 52.8 | 552.5 |

Selected Mice in this table had normal HCT.

As can be seen in Table 5, platelet levels increased significantly after Ad-EPO delivery even if HCT did not increase the first month

This result can be explained by the recombinant erythropoietin presence in blood during the first month after injection, just before the production of anti-EPO antibodies. During the first month recombinant EPO is produced following the injection of the recombinant adenovirus encoding the mutated form, but still functional in mice. This delivered EPO is sufficient to activate megacaryocytopoiesis and induce the platelets differentiation. The binding of the recombinant erythropoietin on the EPO receptor on CFU-MK induced a production of platelets. This result confirms our in vitro observation.

Red blood cell production was not privileged as seen by HCT values but platelets production (platelets values in table 5). Furthermore, it can be observed in table 4, that when HCT stay at a high level, platelets remained at significant higher levels When high level EPO production is maintained, slight but significant platelets increased is observed

Table 6 shows that all TPO−/− mice with hematocrit values below 10%, presented platelets level less than basal platelet values (Normal platelet range is calculated in Ad-Null injected TPO−/−mice 191.33 (±10.78).10³ PLA/mm3). 8 out of the 9 mice in this situation had a decreased platelet levels between 30 to 50%, among them 5 out the 9 had around 50% of their basal platelet values.

TABLE 6
Blood numeration of TPO−/− mice with a HCT less than
10% from the second month (measure 2^{nd} to 3^{rd} month).

| | | Ratio = |
|---|---|---|
| HCT | platelets | Basal platelets values/ |
| (%) | (10³/mm³) | decrease platelets |
| 3.6 | 77 | 2.5 |
| 4.2 | 52 | 3.7 |
| 4.2 | 68 | 2.8 |
| 4.8 | 126 | 1.5 |
| 5.3 | 109 | 1.8 |
| 6.2 | 89 | 2.1 |
| 6.5 | 130 | 1.5 |
| 8.2 | 100 | 1.9 |
| 8.6 | 197 | 1 |

| | |
|---|---|
| Mean values (10³ | 93.875 |
| PLA/mm³ | |
| SD (±10³ PLA/mm³) | 11.68 |

Platelets drop is significant (p = 0.0006 t test) by comparing platelets obtained in table 6 to Ad-Null treated TPO−/− mice.

In addition, as shown in Table 7, when platelet levels are below 100.10³ PLA/mm³, all mice had very low hematocrit levels, or fast EPO-neutralization phenotype (platelet levels ranged from 1.9 to 3.7 fold decrease). 6 mice out of 11 are below 10% HCT, 2 between 10%-20% and 3 between 20%-30%. This result confirms the direct correlation between the EPO remaining level and the residual platelets in C57BL6/J TPO−/− mice.

TABLE 7
Blood numeration of TPO−/− mice with a
platelets level less than 100 · 10³ PLA/mm³
(measures from 2^{nd} to 3^{rd} months) t test p < 0.0001

| HCT % | platelets 10e+3/mm3 |
|---|---|
| 4.2 | 52 |
| 12.9 | 63 |
| 4.2 | 68 |
| 3.6 | 77 |
| 25 | 78 |
| 28.1 (9.9 15 days later) | 84 |
| 18.5 (12.9 15 days later) | 87 |
| 30.4 (14.6 15 days later) | 87 |
| 6.2 | 89 |
| 9.9 | 90 |
| 8.2 | 100 |
| Mean values HCT (%) | 13.75 |
| SD (%) | 3.05 |

### 9.2 Hematopoietic Progenitor Cell Assay

To observe a difference on CFU-MK differentiation and growth using EPO as a differentiation factor , bone marrow progenitor cells were cultured from different adult mouse strains (CD1, Balbc/J, C57B16/J), which were not treated by Ad-EPOmix or Ad-Null.

Bone marrow from tibia and femur was collected using a syringe with a 23G needle in a DMEM completed medium (FCS10%, Penicilline/Streptomycine 1%, glutamin 1%). Red cells were lysed using ammonium chloride and progenitors cells were counted.

500 μl of these progenitor cells was added to 2 ml of a methylcellulose based medium (Methocult medium, Methycellulsose Stemcell #3234 80 ml, prepared and stored as described in manufacturer protocol) in order to have a final concentration of 10⁵ cells/ml. Methycellulose was completed with a cocktail of cytokines: mIL3 10 ng/ml (Abcys) mIL6 10 ng/ml (Abcys) mSCF 50 ng/ml (Abcys) hTPO 10 ng/ml (Abcys) rhEPO 3 U/ml (EPREX) at the final concentration.

1 ml of this medium completed with cell preparation was distributed per Petri dishes 35 mm (GREINER BIO-ONE 627102). Cells were incubated 6-8 days at 37° C. 5% CO2 and 95% humidity.

In order to test the influence of EPO on megacaryopoiesis, 50 ng/ml of a mouse recombinant EPO and mouse recombinant SCF were added.

As seen in the different conditions obtained in table 7 and table 8, BFU-E number increased (3 fold increased in C57B16/J) but also CFU-MK, especially in C57B16/J strain where 3.5 colonies were counted with no EPO addition and 8 counted colonies when EPO was added.

TABLE 8
Determination of progenitors numbers in methocult medium.

| | | | | | B-FUE/ |
|---|---|---|---|---|---|
| | | CFU-GM | CFU-GEMM | CFU-MK | C-FUE |
| CD1 | / | 32.5 | 8 | 0 | 0 |
| | EPO | 41 | 10.5 | 1.5 | 0 |
| | SCF | 41.5 | 11.5 | 3 | 3 |
| | EPO + SCF | 38.5 | 15 | 3.5 | 4 |
| BALBC | / | 19 | 6.5 | 0 | 6.5 |
| | EPO | 26 | 4 | 1.5 | 8.5 |
| | SCF | 26.5 | 8.5 | 0 | 9 |
| | EPO + SCF | 21.5 | 6 | 1 | 5 |
| C57BL6 | / | 52 | 6.5 | 3.5 | 2 |
| | EPO | 62 | 11 | 8 | 6 |
| | SCF | 63.5 | 14.5 | 2 | 1.5 |
| | EPO + SCF | 56 | 4.5 | 4 | 5 |

Conditions were duplicated. The values are means between two identical cultures.

1.-20. (canceled)

21. A regime or regimen for inducing thrombopoiesis, comprising the step a) of administering a thus effective amount of erythropoietin (EPO), derivative or agonist thereof, to a cell type selected from the group consisting of multilineage myeloid progenitor cells, megakaryocyte progenitors and megakaryocytes.

22. The regime or regimen as defined by claim 21, for inducing the differentiation of multilineage myeloid progenitor cells (CFU-GEMM) into megakaryocyte progenitor cells, and comprising the step a) of administering a thus effective amount of erythropoietin, derivative or agonist thereof, to multilineage myeloid progenitor cells.

23. The regime or regimen as defined by claim 21, comprising administering a derivative or agonist able to occupy at least one receptor of erythropoietin and has the same effect as erythropoietin on thrombopoiesis.

24. The regime or regimen as defined by claim 21, comprising mediating the function of said derivative or agonist by interaction with the EPO receptor.

25. The regime or regimen as defined by claim 21, further comprising inducing the differentiation of megakaryocyte progenitor cells into megakaryocytes.

26. The regime or regimen as defined by claim 21, comprising inducing the production of platelets from megakaryocyte cells.

27. The regime or regimen as defined by claim 21, further comprising the step b) of administering to said cell type selected from the group consisting of multilineage myeloid progenitor cells, megakaryocyte progenitors and megakaryocytes, a thus effective amount of trombopoietin, derivative or agonist thereof.

28. The regime or regimen as defined by claim 27, wherein said step b) comprises administering to said multilineage myeloid progenitor cells a thus effective amount of trombopoietin, derivative or agonist thereof.

29. The regime or regimen as defined by claim 21, wherein said administering step b) is carried out before, simultaneously or after the administering step a).

30. The regime or regimen as defined by claim 21, carried out in vitro.

31. The regime or regimen as defined by claim 21, for treating and/or preventing thrombocytopenia in a host organism.

32. The regime or regimen as defined by claim 31, wherein said host organism is a vertebrate.

33. The regime or regimen as defined by claim 31, wherein said host organism is a human.

34. The regime or regimen as defined by claim 31, wherein said thrombocytopenia is a disease selected from the group consisting of vitamin B12 or folic acid deficiency, leukemia or myelodysplastic syndromes idiopathic thrombocytopenic purpura (ITP), thrombic thrombocytopenic purpura (TTP), haemolytic-uremic syndrome (HUS), systemis lupus erythematosus (SLE), cirrhosis, different cancers, hepatic failure, viral infection, HIV and thrombocytopenia medication-induced with anti-cancer drugs, quinine or abciximab.

35. The regime or regimen as defined by claim 34, wherein said thrombocytopenia comprises a bicytopenia.

36. The regime or regimen as defined by claim 34, wherein said thrombocytopenia comprises a single lineage cytopenia.

37. The regime or regimen as defined by claim 21, wherein said effective amount of EPO, derivative or agonist thereof ranges from 2,000 to 20,000 units per month.

38. The regime or regimen as defined by claim 37, wherein said effective amount of EPO, derivative or agonist thereof ranges from 4,000 to 10,000 units per month.

39. The regime or regimen as defined by claim 21, wherein said EPO, derivative or agonist thereof is administered via a technique selected from the group consisting of intravenous injection, intravaginal injection, intrarectal injection, intramuscular injection, subcutaneous, intradermic injection, oral and nasal delivery.

40. The method as defined by claim 21, wherein erythropoietin, derivative, or agonist thereof, or thrombopoietin, derivative, or agonist thereof is formulated for short or long term delivery.

41. The regime or regimen as defined by claim 23, comprising administering a derivative or agonist having the same effect as erythropoietin on differentiation of multilineage myeloid progenitor cells (CFU-GEMM) into megakaryocyte progenitor cells.

1. A method for inducing thrombopoiesis comprising the step a) of administering an effective amount of erythropoietin, derivative or agonist thereof, to a cell type chosen in the group consisting of: multilineage myeloid progenitor cells, megakaryocyte progenitors and megakaryocytes.

2. The method according to claim 1, wherein said method is for inducing the differentiation of multilineage myeloid progenitor cells (CFU-GEMM) into megakaryocyte progenitor cells, said method comprising the step a) of administering an effective amount of erythropoietin, derivative or agonist thereof, to multilineage myeloid progenitor cells.

3. The method according to claim 1 or 2, wherein said derivative or agonist is able to occupy at least one receptor of erythropoietin and has the same effect than erythropoietin on thrombopoeisis, in particular on differentiation of multilineage myeloid progenitor cells (CFU-GEMM) into megakaryocyte progenitor cells.

4. The method according to according to any of the previous claims, wherein the function of said derivative or agonist is mediated by interaction with the EPO receptor.

5. The method according to any of the previous claims, wherein said method further induces the differentiation of megakaryocyte progenitor cells into megakaryocytes.

6. The method according to any of the previous claims, wherein said method induces the production of platelets from megakaryocyte cells.

7. The method according to any of the previous claims, further comprising the step b) of administering to said cell type chosen in the group consisting of: multilineage myeloid progenitor cells, megakaryocyte progenitors and megakaryocytes, an effective amount of trombopoietin, derivative or agonist thereof.

8. The method according to claim 7, wherein said step b) consists in administering to said multilineage myeloid progenitor cells an effective amount of trombopoietin, derivative or agonist thereof.

9. The method according to any of the previous claims, wherein said administering step b) can operate before, simultaneously or after the administering step a).

10. The method according to any of the previous claims, wherein said method is an in vitro method.

11. The method according to any of the claims 1 to 8, wherein said method of the invention is a method for treating and/or preventing thrombocytopenia in a subject.

12. The method according to claim 11, wherein said subject is a vertebrate, preferably a mammal.

13. The method according to claim 11, wherein said subject is human.

14. The method according to claim 11, wherein thrombocytopenia is a disease selected in the group comprising vitamin B12 or folic acid deficiency, leukaemia or myelodysplastic syndrome, idiopathic thrombocytopenic purpura (ITP), thrombic thrombocytopenic purpura (TTP), haemolytic-uremic syndrome (HUS), systemis lupus erythematosus (SLE), cirrhosis, different cancers, hepatic failure, viral infection such as HIV and thrombocytopenia medication-induced such with anticancer drugs, quinine or abciximab.

15. The method according to claim 14, wherein thrombocytopenia is a bicytopenia.

16. The method according to claim 14, wherein thrombocytopenia is a single lineage cytopenia.

17. The method according to claim 11, wherein the effective amount of EPO, derivative or agonist thereof is comprised between 2,000 and 20,000 units per month.

18. The method according to claim 17, wherein the effective amount of EPO, derivative or agonist thereof is comprised between 4,000 and 10,000 units per month.

19. The method according to claim 11, wherein EPO, derivative or agonist thereof is administered via a technique selected in the group consisting in intravenous injection, intravaginal injection, intrarectal injection, intramuscular injection, subcutaneous, intradermic injection, orale and nasal delivery.

20. The method according to claim 11, wherein erythropoietin, derivative, or agonist thereof and eventually thrombopoietin, derivative, or agonist thereof is formulated for short or long term delivery.
